(19) United States
(12) Patent Application Publication
(10) Pub. No.: US 2013/0226263 A1
(43) Pub. Date: Aug. 29, 2013

(54) SYSTEMS AND METHODS OF GATHERING DATA AT THE TIME OF PARAMETER OVERRIDES

(71) Applicant: Cardiac Pacemakers, Inc., (US)

(72) Inventors: Jonathan H. Kelly, Woodbury, MN (US); James O. Gilkerson, Stillwater, MN (US); Kenneth P. Hoyme, Plymouth, MN (US); James R. Kalgren, Lino Lakes, MN (US); David L. Perschbacher, Coon Rapids, MN (US)

(73) Assignee: CARDIAC PACEMAKERS, INC., St. Paul, MN (US)

(21) Appl. No.: 13/774,214

(22) Filed: Feb. 22, 2013

Related U.S. Application Data
(60) Provisional application No. 61/602,928, filed on Feb. 24, 2012.

(51) Int. Cl. A61N 1/372

(52) U.S. Cl. CPC A61N 1/37264
USPC 607/59

(57) ABSTRACT

The current technology is relevant to a system having a programming device in communication with an implantable medical device, an implantable sensor, and electronic medical records. A user interface is in communication with the programming device, and the user interface is configured to receive an override parameter and override rationale.

This application is a non-provisional application claiming priority to U.S. Provisional Application No. 61/602,928, filed Feb. 24, 2012, the entire contents of which is incorporated herein by reference.

### FIELD OF THE INVENTION

The current technology generally relates to programming of medical devices. More specifically, the current technology relates to systems and methods of gathering data at the time of parameter overrides by physicians in implantable medical device (IMD) programming.

### BACKGROUND

Implantable medical devices can be used to provide pacing therapy to patients who have cardiac rhythm problems. For example, an implanted cardiac rhythm management (CRM) device can be used to provide pacing therapy to a patient with sinus node dysfunction, where the heart fails to properly initiate depolarization waves, or an atrioventricular conduction disturbance, where the conduction of depolarization waves through the heart tissue is impaired.

Currently, patient management systems can provide system recommendations of programming parameters based on patient-specific medical data including sensor data. Clinicians generally can override the programming parameter suggestions by the system with little effect on the system. For example, a clinician can be aware of particular symptoms of the patient that the patient management system is unable to detect. The clinician may not be aware of a factor associated with the system recommendations.

Therefore, there is a need for an approach of overriding system-suggested programming parameters that results in updating the system to incorporate new data based on the override parameter and notifies a clinician of an override parameter that is inconsistent with patient indications.

### SUMMARY OF THE INVENTION

In one embodiment the current technology is relevant to a method where parameters associated with a medical device are displayed. Medical device data and an override parameter is received. A notification is displayed that the override parameter is inconsistent with patient indications. The user is prompted for override rationale associated with the override parameter.

In another embodiment, the current technology is relevant to a system having a programming device in communication with an implantable medical device, an implantable sensor, and electronic medical records. A user interface is in communication with the programming device, and the user interface is configured to receive an override parameter and override rationale. The programming device is configured to incorporate the override parameter and override rationale in system operations.

### BRIEF DESCRIPTION OF THE DRAWINGS

The invention may be more completely understood and appreciated in consideration of the following detailed description of various embodiments of the invention in connection with the accompanying drawings.

FIG. 1 is a schematic diagram of an exemplary implementation of a cardiac rhythm management (CRM) system, including an implanted CRM device, a programming device, and a patient management computer system, consistent with at least one embodiment of the technology disclosed herein.

FIG. 2 is a schematic diagram of an exemplary implementation of a cardiac rhythm management system where the programming device is remote from the patient having the implanted CRM device.

FIG. 3 is a flow chart depicting one method consistent with the technology disclosed herein.

FIG. 4 is an example screenshot of indications consistent with the technology disclosed herein.

FIG. 5 is an example screenshot of an application of an override parameter consistent with the technology disclosed herein.

FIG. 6 is an example screenshot of a notice consistent with the technology disclosed herein.

FIG. 7 is an example screenshot of conflicting system parameters consistent with the technology disclosed herein.

FIG. 8 is an example screenshot of user options for resolving conflicting parameters consistent with the technology disclosed herein.

FIG. 9 is an example screenshot of selecting “physician preference” to resolve conflicting parameters, consistent with the technology disclosed herein.

FIG. 10 is an example screenshot of selecting “other” to resolve conflicting parameters, consistent with the technology disclosed herein.

FIG. 11 is an example screenshot of a user input interface to enter user rationale, consistent with the technology disclosed herein.

FIG. 12 is an example screenshot of entered user rationale to resolve conflicting parameters, consistent with the technology disclosed herein.

FIG. 13 is a flow chart consistent with an example implementation of the technology disclosed herein.

FIG. 14 is a flow chart consistent with an example implementation of the technology disclosed herein.

FIG. 15 is a schematic diagram of an implementation of the components of a programming device or user interface, in accordance with various embodiments.

FIG. 16 is a schematic view of components of an implantable medical system in accordance with an embodiment of the invention.

### DETAILED DESCRIPTION

The technology disclosed herein relates generally to a medical device system that formulates operation parameters based on data available to it. The system allows a caretaker, physician, or other system user to override the system parameters and displays a warning if the override parameter is inconsistent with patient indications. The user can enter their own rationale for overriding the system parameters. The system can apply the override parameter, the override rationale, or both to future system operations and diagnosis. In one embodiment, the system disallows user override of system parameters. While this technology can be applied to a variety of medical systems, including non-implantable devices, the implementation described herein will be with regard to implantable medical devices, particularly cardiac devices. Those having skill in the art will recognize technology applicability to devices such as insulin pumps, stimulators, and non-cardiac devices generally.

### Medical Device Details

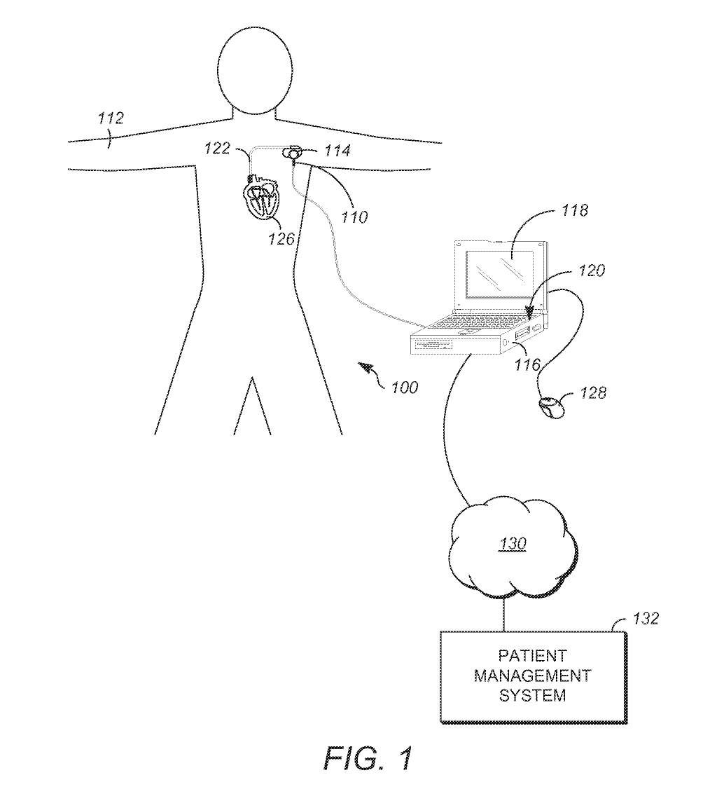

FIG. 1 is a schematic of an exemplary cardiac rhythm management (CRM) system 100, consistent with at least one embodiment of the technology disclosed herein. The system 100 can include an implantable medical device 114 disposed within a patient 112. The implantable medical device 114 can include pacing functionality. The implantable medical device 114 can be of various types such as, for example, a pacemaker, a cardioverter-defibrillator, a cardiac resynchronization device, or the like. In some embodiments, the implantable medical device 114 can include one or more leads 122 disposed in or near the patient's heart 126.

The implantable medical device 114 can include one or more implantable sensors in order to gather patient 112 data. For example, the implantable medical device 114 can include an activity level sensor, a respiration sensor, a blood pressure sensor, and/or other sensors.

### Programming Device Details

The implantable medical device 114 can be in communication with a programming device 116 or user interface. The programming device 116 is also in communication with the implantable sensor of the implantable medical device 114, and/or one or more other implantable sensors. In some embodiments, communication between the implantable medical device 114 and the programming device 116 can be via inductive communication through a wand 110 held on the outside of the patient 112 near the implantable medical device 114. However, in other embodiments, communication can be carried out via radiofrequency transmission, acoustically, or the like. The implantable medical device 114 can be configured to store data over a period of time and periodically communicate with the programming device 116 in order to transmit some or all of the stored data.

The programming device 116 can be for example, a programmer, a programmer/recorder/monitor device, a computer, an advanced patient management system, a personal digital assistant (PDA), or the like. A programming device is one example of a user interface. As used herein, the term programming device 116 refers to a device that programs implanted devices and records data from implanted devices. The programming device may also allow monitoring of the implanted device. Exemplary programmer/recorder/monitor devices include the Model 3120 Programmer, available from Boston Scientific Corporation, Natick, Mass. The programming device 116 can include a user interface such as a keyboard 120, a mouse 128, a touch screen, or more than one such device to receive user input. The programming device 116 can also include a video output channel and a user interface such as a video display 118 for displaying videos, user prompts, device operation parameters, settings, recommendations, and the like. In addition, the video display 118 can also be equipped with a touch screen, making it into a user input device as well.

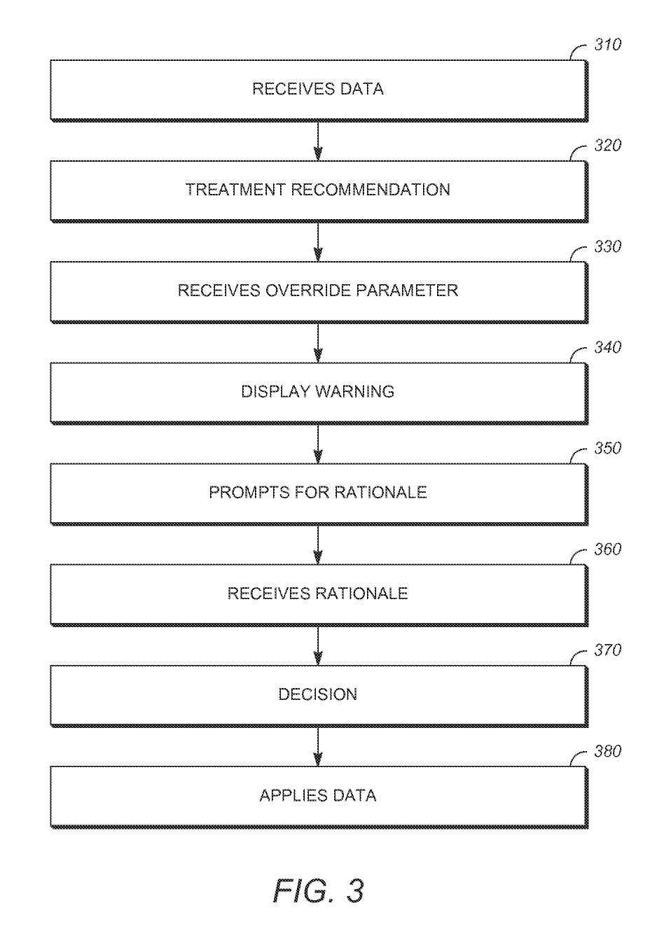

The programming device 116 can display real-time data and/or stored data graphically, such as in charts or graphs, and textually through the user interface screen. Generally, the programming device 116 displays parameters associated with the medical device 114. Parameters associated with the medical device 114 can be device operational parameters, patient indications relevant to the medical device 114, and the like. In at least one embodiment, the parameters associated with the medical device 114 can include system-recommended parameters that are formulated by the system. In addition, the programming device 116 can prompt a user for particular data, such as for an override parameter and override rationale associated with the override parameter, which will be explained in the discussion of FIG. 3, below. The programming device 116 can also display response options for the prompt, such as providing options associated with potential override rationale associated with the override parameter. Notifications that an override parameter is inconsistent with patient indications can also be displayed by the programming device 116.

The programming device 116 can input and store a user's response to the various programming prompts, such as inputting and saving an override parameter or override rationale to exclude conflicting data from system processes, such as excluding conflicting data from received data. The conflicting data can also be excluded by the programming device 116 from the formulation of system-recommended parameters. The programming device 116 can also display indications of system confidence levels relative to particular data or operation parameters based on a variety of factors including sensor reliability, age of a patient's electronic files, past accuracy of the data or operation parameters, and the like. The programming device can also display guidance to a user regarding data accuracy.

In various embodiments, the programming device 116 is in communication with a patient management system 132. The communication link 130 between the programming device 116 and the patient management system 132 may be via phone lines, the Internet, or any other data connection. In another embodiment, the programming device 116 is not in direct communication with a patient management system 132, but can be in indirect communication with the patient management system 132. The patient management system 132 can additionally be in communication with electronic patient medical records in a variety of embodiments.

The programming device 116 is capable of changing the operational parameters of the medical device 114, and is therefore referred to as a programmer. Typically, programmers are used to interface with medical devices in a clinic or hospital setting. In this context, the user of the programming device 116 is a clinician, physician or trained technician.

### Remote Programming Embodiment

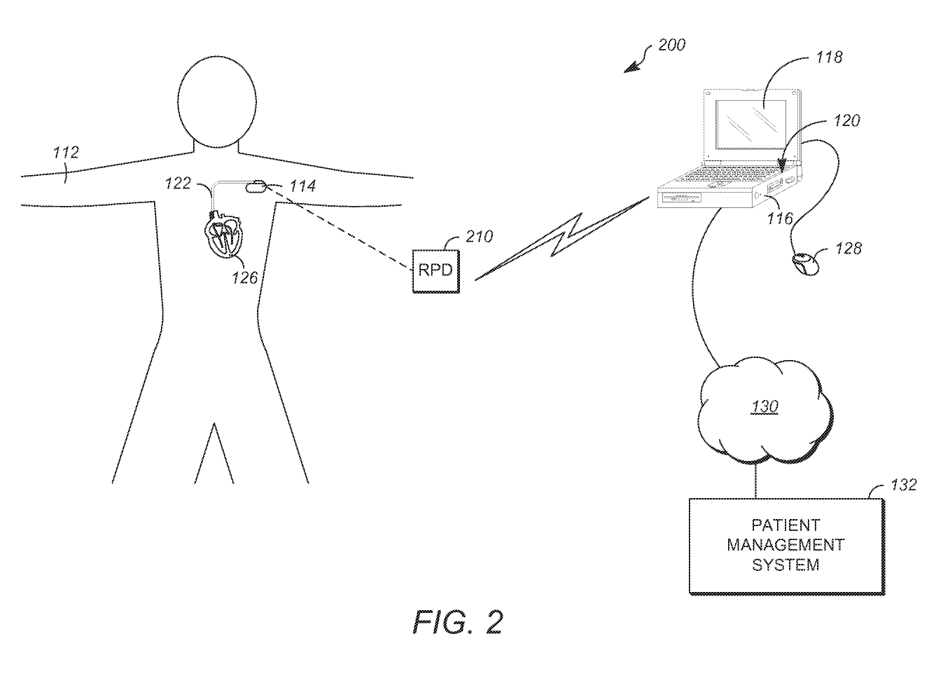

Now referring to FIG. 2, a CRM system 200 is illustrated which is designed for use when the programming device 116 and the patient 112 are in different locations, so that the programming device 116 is remote from the patient 112 and not physically present in the same space as the patient 112. For example, the patient 112 may be at his or her home while the clinician is at a hospital which is a few miles away or hundreds of miles away. Like reference numbers between FIG. 1 and FIG. 2 indicate like elements. In the CRM system 200 of FIG. 2, a remote programming device 210 is in the patient's 112 location and establishes communication with the implantable medical device 114. Communication between the remote programming device 210 and the implantable medical device 114 can be carried out by radiofrequency transmission, acoustically, or by inductive communication using a wand held on the outside of the patient 112 near the device 114.

The remote programming device 210 is in communication with a local programming device 116. The communication link 230 between the local programming device 116 and the remote programming device 210 may be via phone lines, the Internet, or any other data connection. Other details of the programming device 116 and the implantable medical device 114 are similar to as described with respect to FIG. 1.

### Method Description

FIG. 3 is a flow chart depicting one method consistent with the technology disclosed herein. The system receives data 310, makes a treatment recommendation 320, receives an override parameter 330, displays a notice 340, prompts the user for rationale 350, receives override parameter rationale 360, decides 370, and applies the data 380.

Generally, the system can receive data 310 from a variety of sources. An implantable medical device and any implantable sensors can provide data including device operation parameters, patient indications, event counters, and any other data from the implantable medical device and/or from the implantable sensors. The system can receive data from electronic medical records. The electronic medical records can include patient medical records, historical implantable medical device operation parameters, indications read or calculated from electronic charts and electronic medical records, user-entered indications, episodes, and trends in patient indications, as examples. The system can also receive data from a caretaker. Such data can include symptom data, operation parameters, and the like.

The system makes a treatment recommendation 320 that conveys device operation parameters. Generally, the system makes a treatment recommendation based on data available to the system. The treatment recommendations will generally be consistent with recent patient indications and other data available to the system. The treatment recommendations can be communicated to a system user through a user interface, such as displayed on a screen. Other parameters associated with the medical device and the treatment recommendations can also be communicated to a system user. Each treatment recommendation can have a system confidence level associated therewith.

The system confidence level can be based on a variety of factors associated with the data, such as the reliability of a data source, historical patient data including event occurrences, past incorrectness of treatment recommendations, conflicting data, and so on. In a variety of embodiments the system provides an indication of the system confidence level of the treatment recommendations to the user, which can be expressed in a variety of ways including a percentage or number value, color code, text and so on. The treatment recommendations can be displayed for the caretaker on a user interface, who may decide to override one or more parameters.

The system can receive an override parameter at step 330, where the override parameter is a data entered in by a user that conflicts to some degree with existing system data. The override parameter can override the system treatment recommendation, for example. In such an embodiment the override parameter is one or more new operation parameters, such as a new course of treatment. The override parameter can also override patient indications, as another example.

The system can receive an override parameter through a variety of user interface devices such as a keyboard, touchscreen, mouse, microphone, combinations thereof, and so on. In a variety of embodiments the system is configured to determine whether the received override parameter is consistent with patient indications. Such a determination can include identifying conflicting system data based on the override parameter.

If the override parameter conflicts with system data, the system displays a notice 340 to provide information to the user. The notice can be a warning indicating that the chosen treatment is not consistent with patient indications. The warning can also notify the system user of data that is in conflict with the override parameter, or provide another explanation of the inconsistency of the override parameter. The system can also provide the user with rationale associated with the recommended parameter.

The system is configured to prompt the user for override rationale associated with the override parameter at step 350. The override rationale corresponds to the reason that the user entered an override parameter into the system. In at least one embodiment, the system prompt provides a selection of two to six options of which the user can choose the one that most closely represents the rationale of the user. In at least one embodiment, the system prompt provides an open field within which a user can provide the override rationale.

The system then receives override rationale at step 360 associated with the override parameter. In multiple embodiments the system stores the received override rationale, as well. In at least one embodiment, the override rationale can simply indicate user preference. The override rationale can also indicate inadequacy of a sensor. For example, override rationale can indicate that particular sensor data is incorrect because of a broken sensor lead or that the sensor is too sensitive. In some embodiments the override rationale can indicate that past treatments by the system were inappropriate. In some embodiments the override rationale can indicate a patient symptom that is not observable by the system. For example, the override rationale can indicate that the patient exhibits or describes symptoms not sensed by the system such as inactivity or shortness of breath.

Based on the override parameter and the override rationale, the system decides at step 370 whether to allow or disallow the system override or, in at least one embodiment, whether more information is needed. For example, if the override rationale justifies the override parameter, and is consistent with patient indications, then the system can allow the system override. However, if the override parameter, override rationale, and patient indications are somehow inconsistent, the system can disallow the system override, prompt the user for more information, or display alternate operation parameters. These options will be described in more detail below.

The system incorporates the override data at step 380, including the override parameter and the override rationale, into system operations. In a variety of embodiments the system is configured to identify conflicting data based on the override data and permanently or temporarily exclude conflicting data from system operations. The system can, for example, suppress reporting an indication based on conflicting data for a particular period of time. In another example, the system can permanently exclude conflicting data from all algorithms and calculations. In at least one embodiment, the system displays data accuracy guidance to the user. Data accuracy guidance can provide the user with suggestions for improving data accuracy such as detection enhancements.

### Example Implementations of the Method of FIG. 3

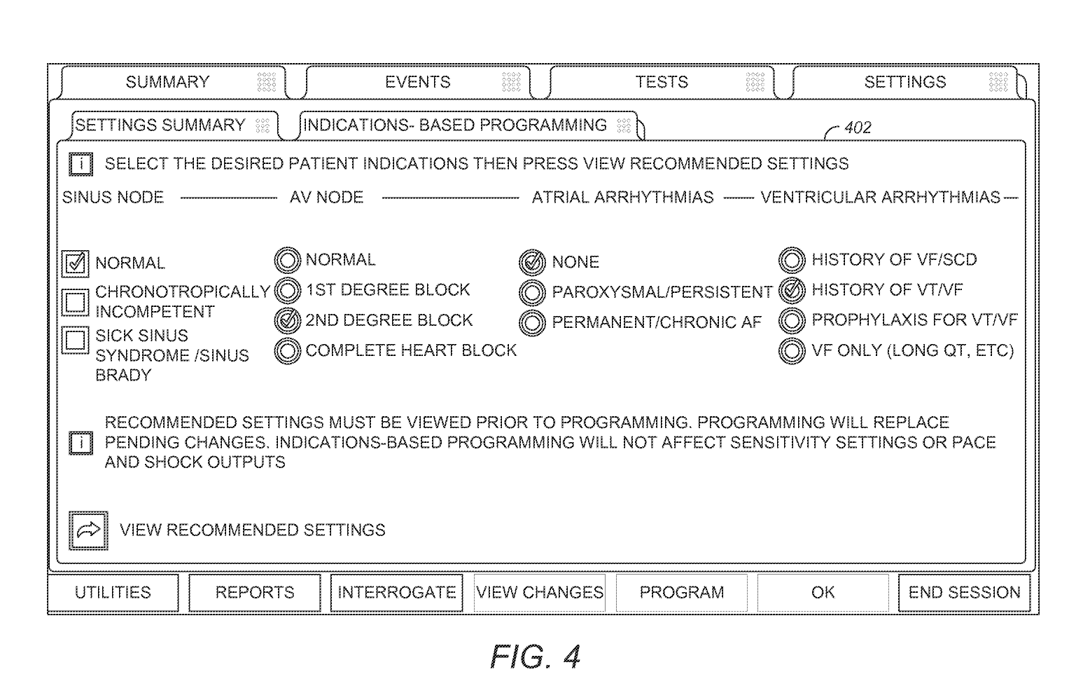

FIGS. 4-14 are consistent with example implementations of the technology disclosed herein. FIGS. 4-12 are example screen shots from a system implementing a method consistent with FIG. 3. As described above, the system receives data, makes a treatment recommendation, receives an override parameter, displays a notice, prompts the user for rationale, receives override parameter rationale, and stores conflicting data.

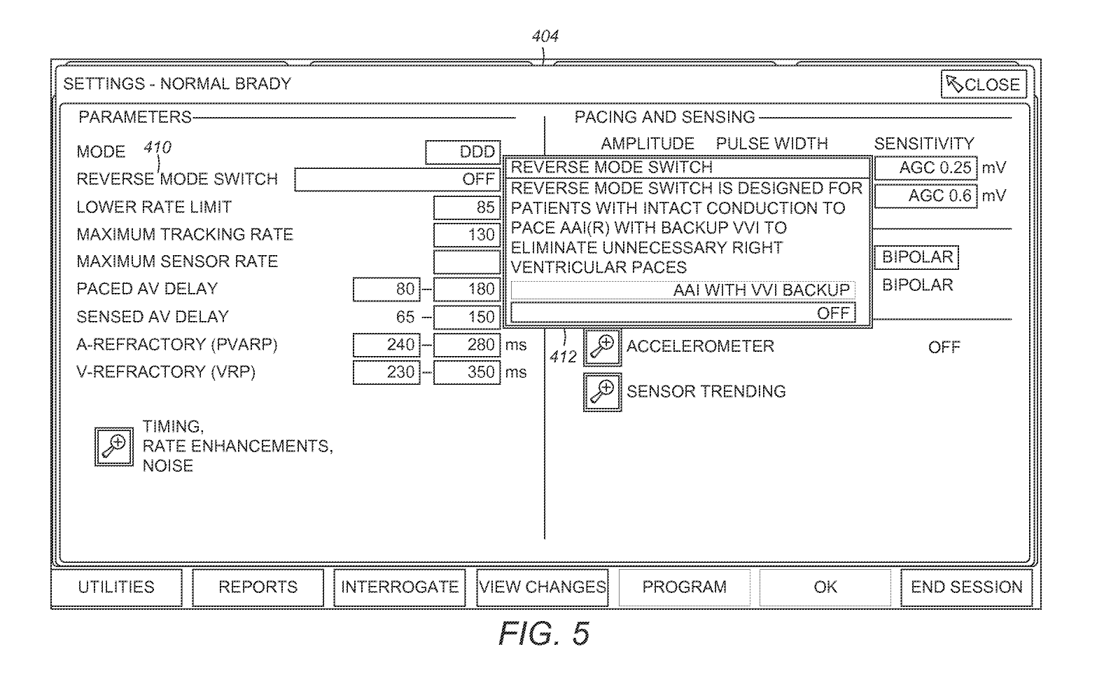

The screenshot of FIG. 4 depicts the patient indications 402 based on data received by the system. Examples of patient indications identified by the system include at least sinus node conditions including chronotropic incompetence and sick sinus syndrome, atrioventricular blocks including a first degree block, second degree block, or a complete heart block, atrial arrhythmias including paroxysmal and chronic, and data associated with ventricular arrhythmias. Based on the patient indications 402 the system provides a treatment recommendation 404 as shown in the “Settings—Normal Brady” screen of FIG. 5.

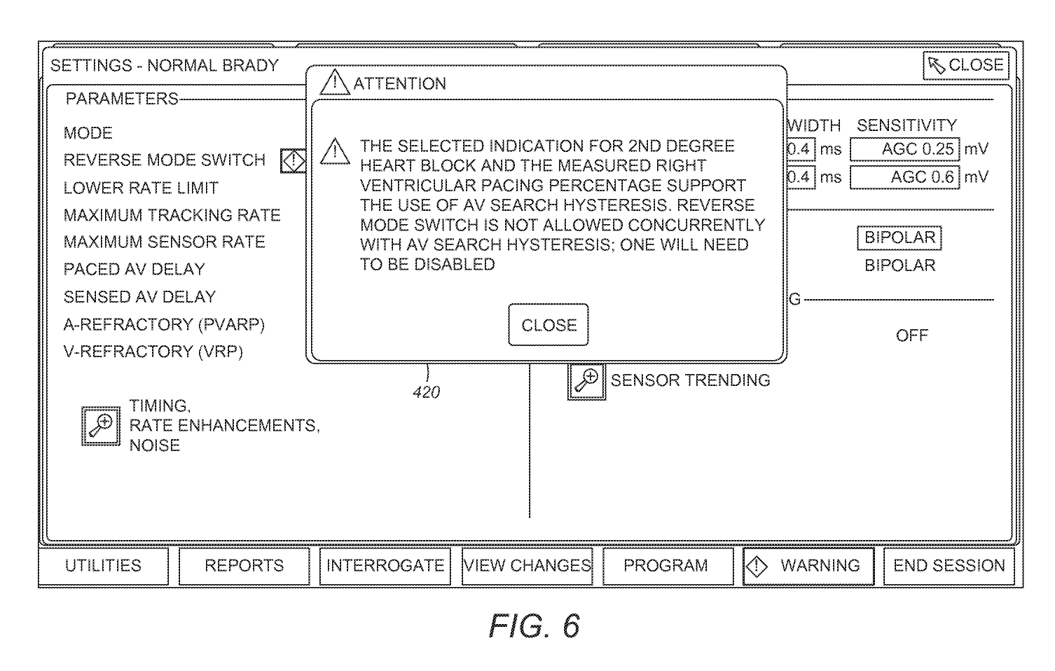

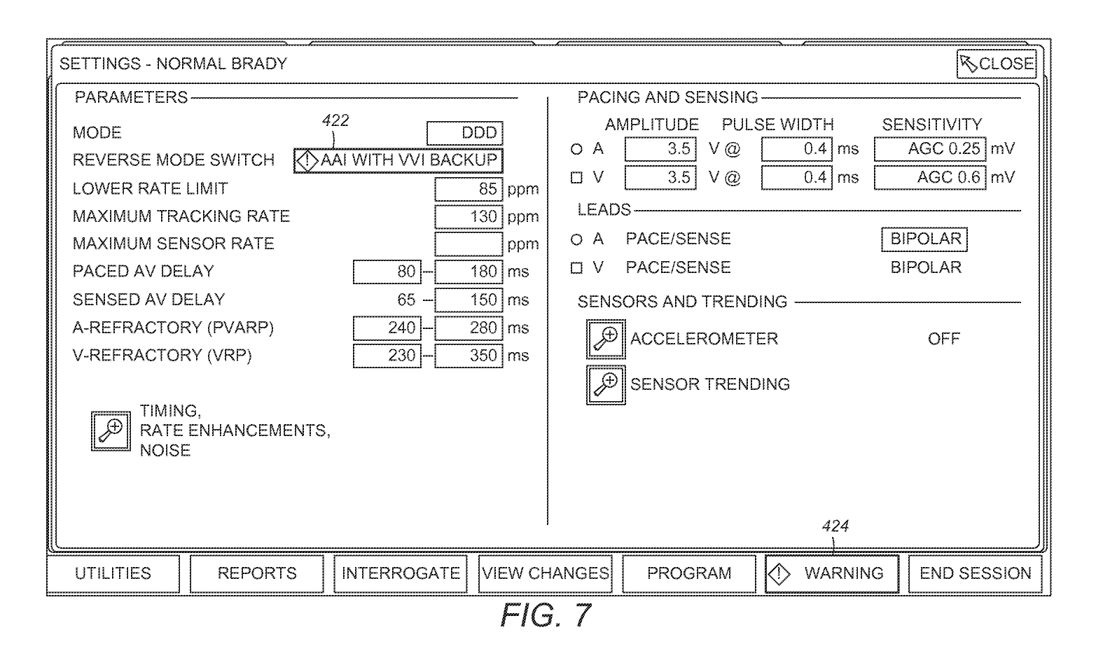

In FIG. 5 the user attempts to override system recommendations and enable a reverse mode switch feature 410 to “AAI with VVI Backup” from an “off” position, which brings up a notification screen 412 explaining the reverse mode switch feature. In FIG. 6 a pop-up notification 420 provides notice of a conflict between enabling the reverse mode switch and the patient indications. In FIG. 7 the override parameter 422 (the reverse mode switch) that presents a conflict is depicted to communicate a warning to the system user through the use of a relatively contrasting color, such as red, and an exclamation point. The color red, or another color, can be consistently associated with parameters that the system has low confidence in and/or parameters that a user must address before continuing the session. For example, in the current situation the system does not allow the system user to program the current settings because of the conflicting parameters. A “warning” button 424 allows the user to access the conflicting parameters to resolve the conflict. In at least one other embodiment, the pop-up notification 420 depicted in FIG. 6 can provide a direct mechanism for the system user to resolve the conflict.

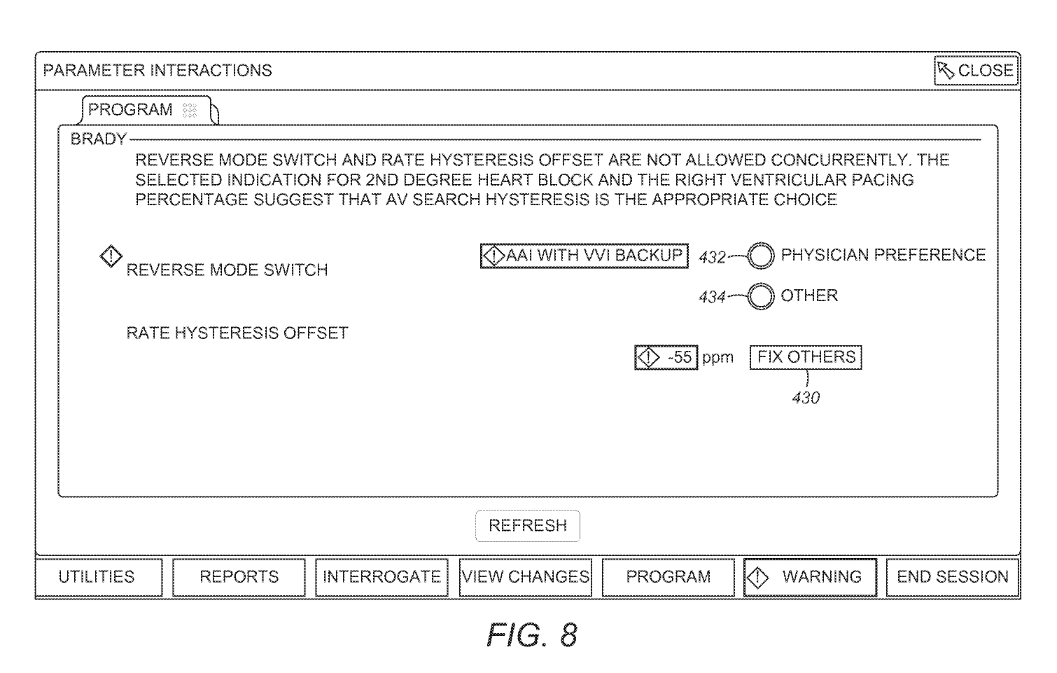

In FIG. 8 the system user is invited to resolve the conflicting parameters. By selecting the “fix others” button 430 the system resolves the conflict based on system data which, in this scenario, would disable the reverse mode switch. The system user can also select a “physician preference” radio button 432 which provides the system with the user rationale for the setting, and an “other” radio button 434, which will be discussed in more detail, below.

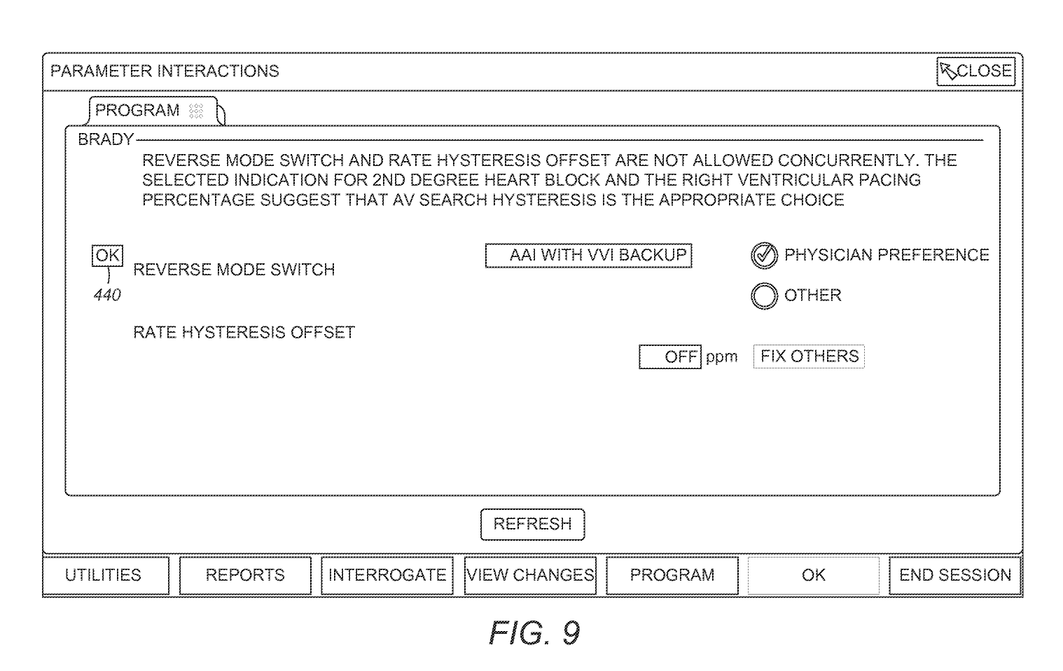

In FIG. 9 the “physician preference” radio button 432 has been selected by a system user. The original system parameter (AV Search Hysteresis) is disabled and the override parameter—the reverse mode switch—is enabled based on user preference. Warning colors and icons are removed to indicate that there is no longer a warning associated with the parameters, and the “warning” button is replaced with an “OK” button 440 to allow system programming to occur. The system can now depict at least the override parameter 422 in the color green, where green is consistently used by the system to indicate high system confidence of the particular parameter. In such an embodiment, the system stores the override parameter 422 and override rationale and applies that data to future system operations and inquiries, and can additionally provide such data to other systems with which it may directly or indirectly interface including electronic clinician notes, electronic medical records, remote monitoring systems, and the like. As such, it can be desirable to store the override parameter and the override rationale with an associated timestamp for future reference.

In another embodiment, the override parameter 422 can be depicted in the color yellow, where yellow consistently is used to indicate a medium system confidence level. In such an embodiment, in future system operations, the yellow presentation of the override parameter 422 can be a reminder that the override parameter 422 previously overrode system indications, for example. In such an embodiment, the override parameter 422 can be applied automatically to future system operations or the system user can be reminded through a prompt to confirm use of the override parameter 422 in each future system operation. In embodiments where the user is prompted for confirmation, it can be desirable to provide the user with data associated with the programming changes, including the user rationale.

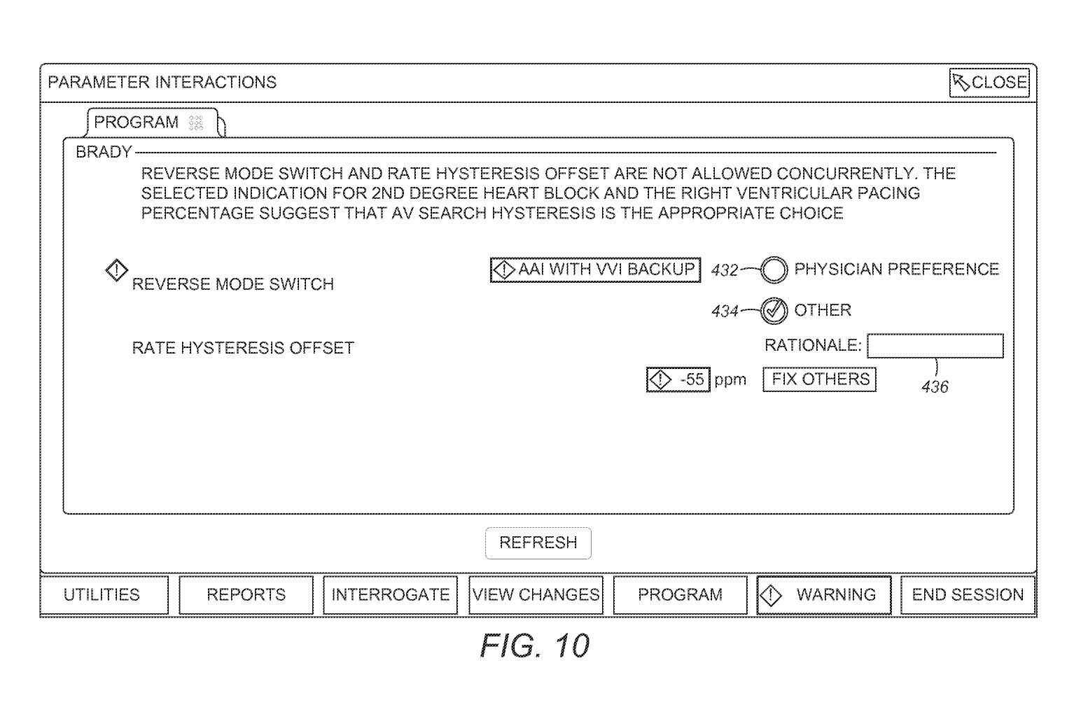

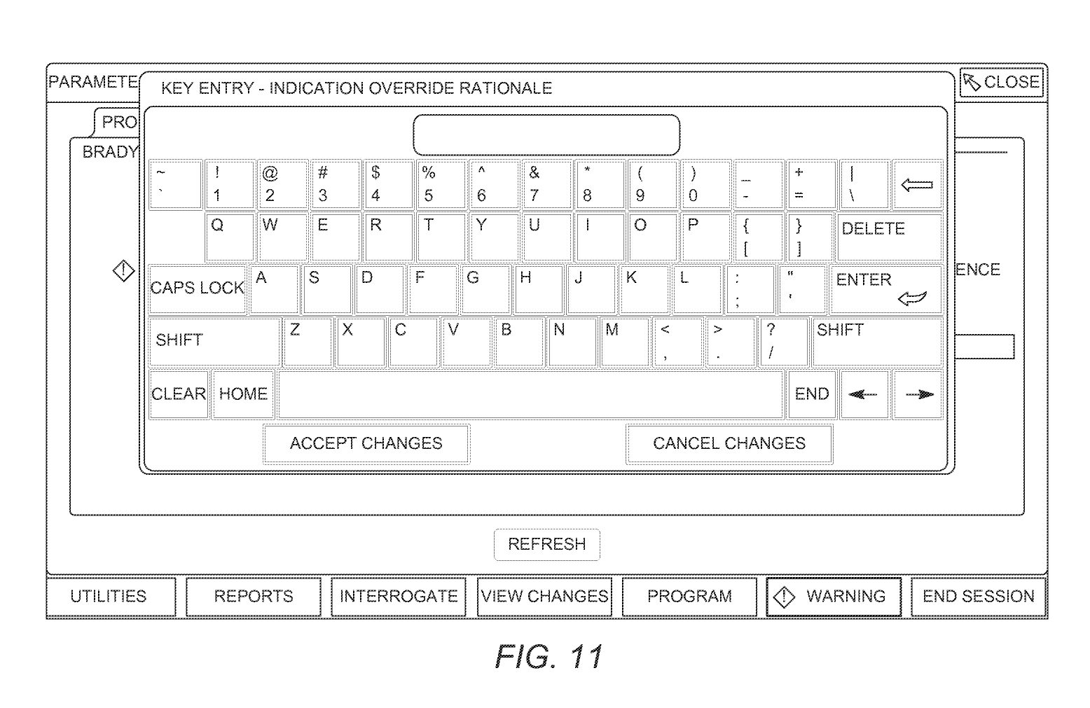

In FIG. 10, the system user has selected the “other” radio button 434 from the screenshot depicted in FIG. 8, which provides the user with a field 436 within which the user can enter in their rationale for the conflicting override parameter 422. In another embodiment, a drop-down selection list can be provided that specifies a plurality of rationales. Those having skill in the art will appreciate that there are a variety of ways a system user can provide the system with their rationale. The override parameter 422 is still depicted as having low system confidence (as described above, in the color red, for example). In an embodiment consistent with FIG. 10, the user can input their rationale through a variety of user interface devices, such as through a touch-screen keyboard, as depicted in FIG. 11.

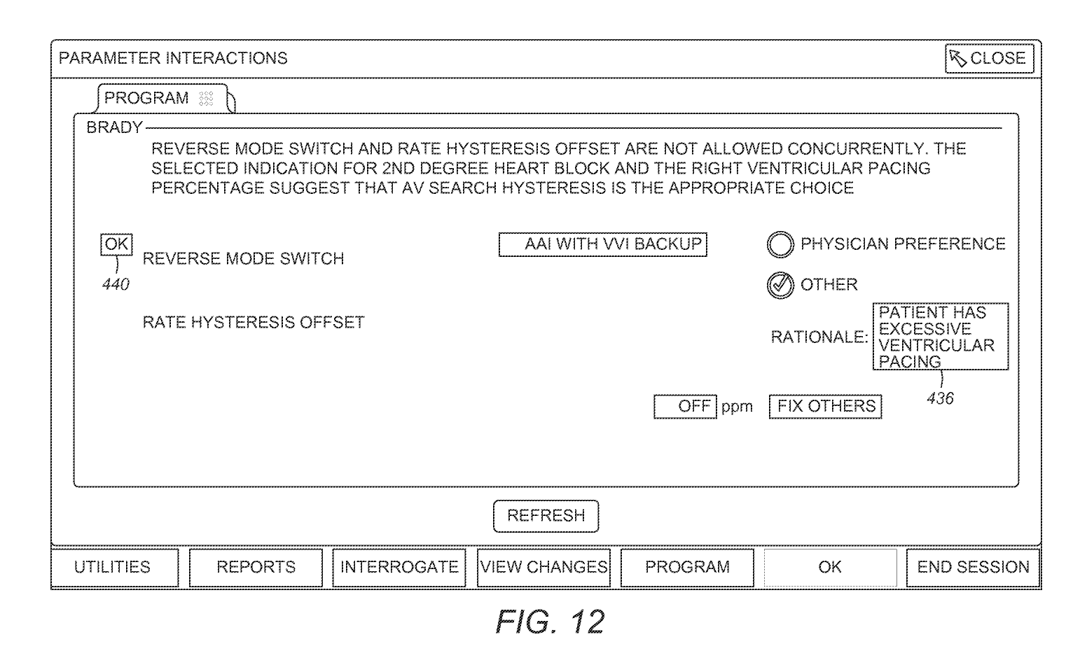

In FIG. 12 the user has entered “Patient has excessive ventricular pacing” in the rationale field 436. The rationale entered by the user can encompass a variety of reasons for the conflicting override parameter including, for example, medication prescriptions, therapies, factors non-observable to a medical device and factors not taken into consideration by the system, such physiological capabilities of the patient.

As another example of free text being entered as override rationale, a system recommends a pacing rate for a patient that has had an ischemic event. If a caregiver believes that the pacing rate is too high for the patient, given the patient's condition, the caregiver could provide an override parameter that reduces the pacing rate and provide override rationale by way of a free text entry communicating the following: “Recommended pacing rate too high due to ischemia.”

Returning back to FIG. 12, the conflicting system parameter (AV Search Hysteresis) is disabled and the reverse mode switch is enabled based on user preference. Warning colors and icons are removed to indicate that there is no longer a warning associated with the parameters, and the “warning” button is replaced with an “OK” button 440 to allow system programming to occur. The override parameter 422 can be displayed to indicate high system confidence in view of the user having provided rationale. As described above, high system confidence can be associated with the color green, where green is consistently used by the system to indicate high system confidence of the particular parameter. The override parameter 422 and the user rationale 436 can be saved by the system and provided during future system use.

As described above, in at least one embodiment, the system can be configured to communicate medium system confidence in situations where an initial programming parameter is overridden, such as in the current example. As such, the override parameter 422 can be depicted consistently with how the system communicates medium system confidence, such as in the color yellow. This can be a reminder to system users that the override parameter 422 overrode another parameter. With medium system confidence, the system can be configured to either automatically apply the override parameter 422 in future system use or prompt the user to take affirmative action to apply the override parameter 422 to each future system use. Those having skill in the art will appreciate a variety of ways that such an override parameter 422 can be applied to the system.

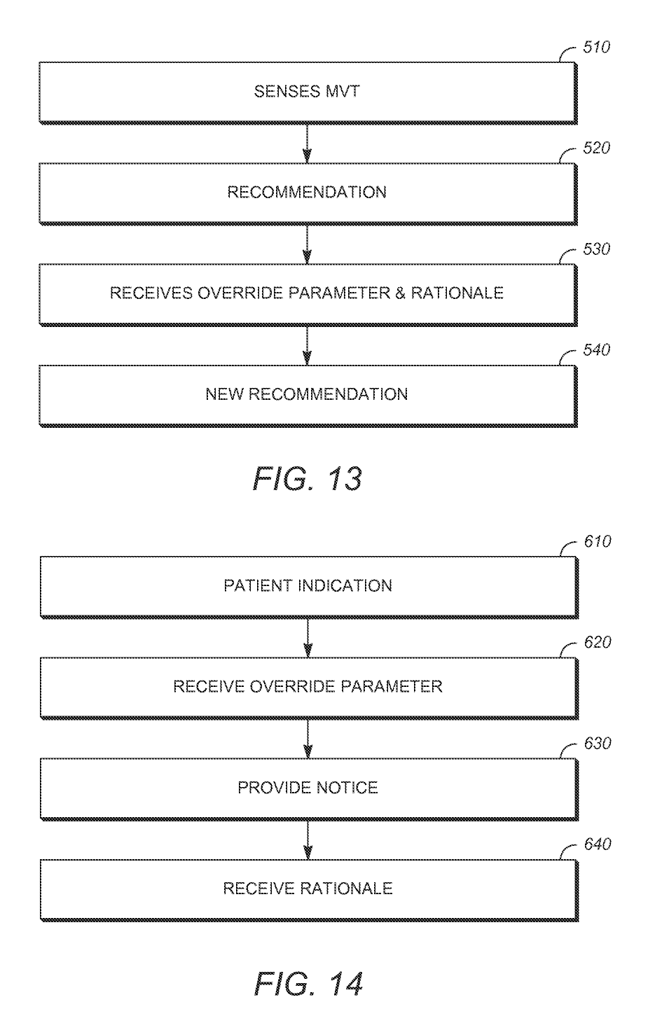

FIG. 13 is a flow chart consistent with an example implementation of the technology disclosed herein. The system senses 510 what is believed to be monomorphic ventricular tachycardia (MVT). Based on this, the system recommends 520 turning on antitachycardia pacing (ATP). ATP is pacing at a faster rate than the ventricular tachycardia (VT), and is often successful in terminating the VT. ATP can additionally be used for VT episodes that are relatively slow compared to ventricular fibrillation. If ATP is not successful within a few cycles, then the capacitors are charged for a defibrillating shock. If ATP is successful, the painful shock is avoided.

However, in response to the recommendation 520 that ATP is turned on, the physician provides an override parameter and rationale 530 based on the physician's understanding that the patient event was supraventricular tachycardia (SVT). The physician can provide the override parameter in a variety of ways. For example, the physician can turn off ATP or the physician can override the MVT reading of the system by inputting SVT in the system. The physician can then provide override rationale based on the override parameter. Where the physician turns off ATP, the override rationale can be the occurrence of SVT rather than MVT. Where the physician overrides the MVT reading with SVT, the override rationale can be the origin of the electrical signal, for example. These two rationales, along with others, can be provided in a list for the physician to select one or both. The physician can also be presented with a text box for entering the rationale.

Based on the override parameter and rationale provided, the system applies the data to provide a new recommendation 540. In the current example, the system can recommend turning on detection enhancements instead, where the detection enhancement protocols can help the device distinguish between MVT and SVT.

FIG. 14 is a flow chart consistent with another example implementation of the technology disclosed herein. A patient has an indication 610 programmed as “VT/VF” indicating that the patient suffers from episodes of both ventricular tachycardia (VT) and ventricular fibrillation (VF). This can be based on a variety of data including the fact that the system has treated the patient for episodes of VT in the past with pacing or a defibrillating shock. If the physician attempts to override 620 the patient indication from “VT/VF” to “VF-only”, the system will provide notice 630 to the physician of the inconsistency. For example, the notice 630 can communicate that the device has treated this patient for VT and the change from a VT/VF indication to a VF-only indication is inconsistent with the past treatment.

The system can then prompt the user to either provide override rationale or revert back to the initial patient indication. Either through entering text or selecting an option, the physician can specify override rational 640 communicating the belief that the device inappropriately treated VT in the past for this patient. The system can query the physician for supporting facts regarding the inappropriate treatment.

### Programmer Hardware

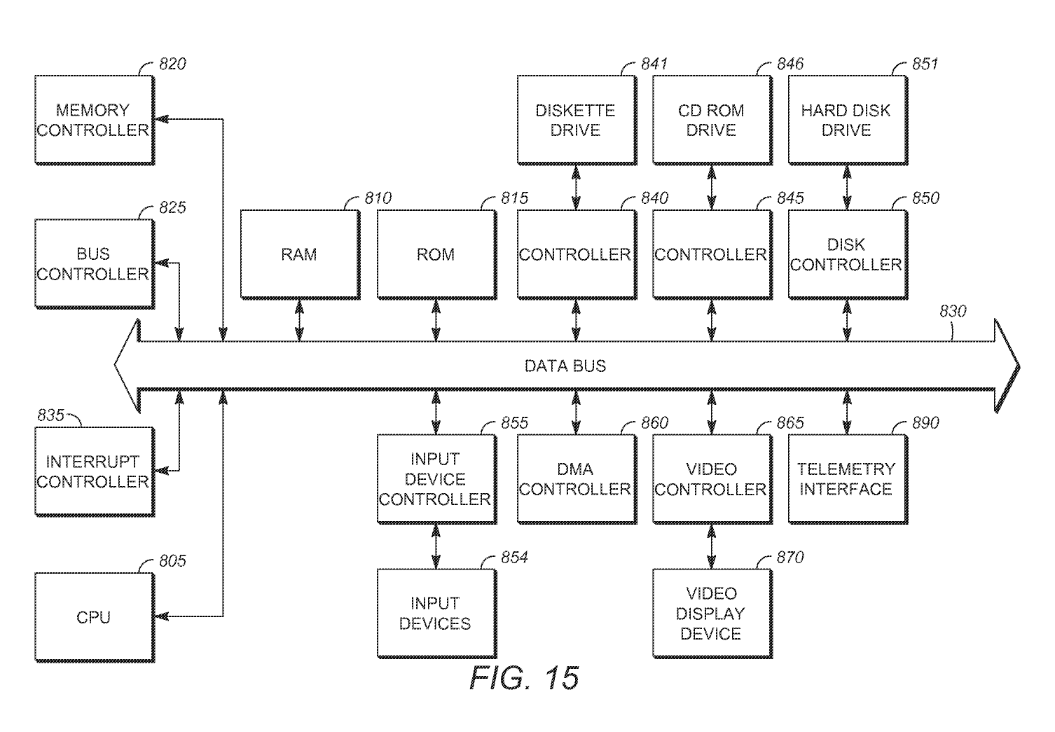

Programming devices can include components common to many computing devices. Referring now to FIG. 15, a diagram of various components is shown in accordance with some embodiments of the invention. However, it is not required that a programming device have all of the components illustrated in FIG. 15.

In one embodiment, the programming device includes a central processing unit (CPU) 805 or processor, which may include a conventional microprocessor, random access memory (RAM) 810 for temporary storage of information, and read only memory (ROM) 815 for permanent storage of information. A memory controller 820 is provided for controlling system RAM 810. A bus controller 825 is provided for controlling data bus 830, and an interrupt controller 835 is used for receiving and processing various interrupt signals from the other system components.

Mass storage can be provided by diskette drive 841, which is connected to bus 830 by controller 840, CD-ROM drive 846, which is connected to bus 830 by controller 845, and hard disk drive 851, which is connected to bus 830 by controller 850. User input to the programmer system may be provided by a number of input devices 834. For example, a keyboard, touch screen, mouse, or more than one of these, can connected to bus 830 by input device controller 855. DMA controller 860 is provided for performing direct memory access to system RAM 810. A visual display is generated by a video controller 865 or video output, which controls video display 870. The external system can also include a telemetry interface 890 or telemetry circuit which allows the external system to interface and exchange data with an implantable medical device. It will be appreciated that some embodiments may lack various elements illustrated in FIG. 15.

### Implantable Device Hardware

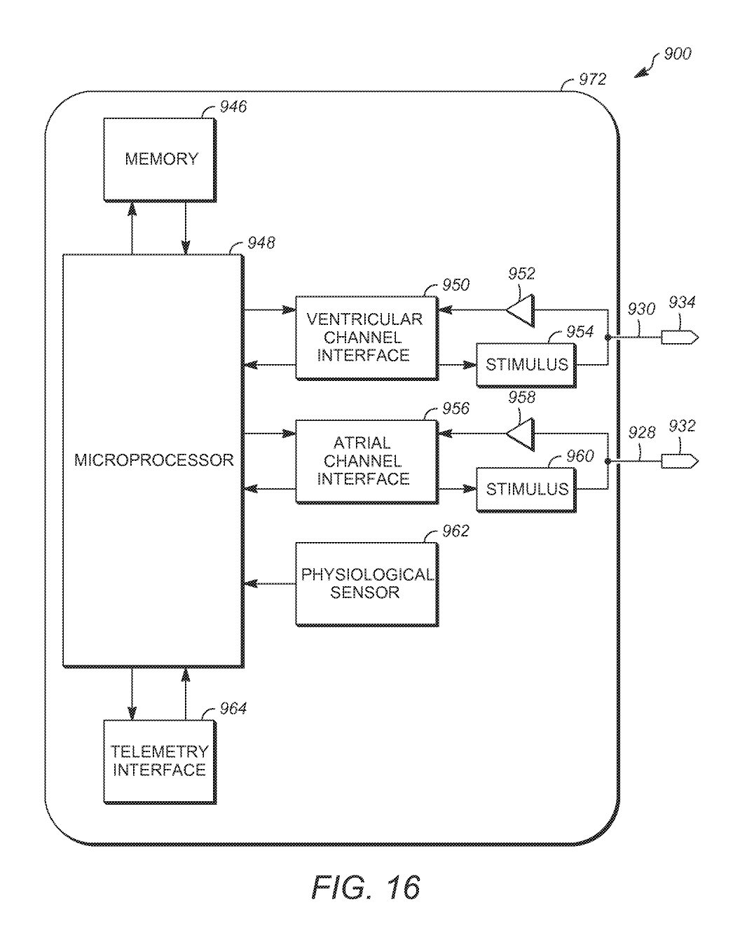

Referring now to FIG. 16, some components of an exemplary implantable system 900, such as an implantable CRM device, are schematically illustrated. The implantable medical system 900 can include an implantable medical device 972 coupled to one or more stimulation leads 930 and 928. The implantable device 972 can also include other sensors such as an activity sensor 962.

The implantable device can include a microprocessor 948 (or processor) that communicates with a memory 946 via a bidirectional data bus. The memory 946 typically comprises ROM or RAM for program storage and RAM for data storage. The implantable device can be configured to execute various operations such as processing of signals and execution of methods as described herein. A telemetry interface 964 is also provided for communicating with an external unit, such as a programmer device or a patient management system.

The implantable device can include ventricular sensing and pacing channels comprising sensing amplifier 952, output circuit 954, and a ventricular channel interface 950 which communicates bidirectionally with a port of microprocessor 948. The ventricular sensing and pacing channel can be in communication with stimulation lead 930 and electrode 934. The implantable device can include atrial sensing and pacing channels comprising sensing amplifier 958, output circuit 960, and an atrial channel interface 956 which communicates bidirectionally with a port of microprocessor 948. The atrial sensing and pacing channel can be in communication with stimulation lead 928 and electrode 932. For each channel, the same lead and electrode can be used for both sensing and pacing. The channel interfaces 950 and 956 can include analog-to-digital converters for digitizing sensing signal inputs from the sensing amplifiers and registers which can be written to by the microprocessor in order to output pacing pulses, change the pacing pulse amplitude, and adjust the gain and threshold values for the sensing amplifiers.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as “arranged”, “arranged and configured”, “constructed and arranged”, “constructed”, “manufactured and arranged”, and the like.

One of ordinary skill in the art will understand that the modules, circuitry, and methods shown and described herein with regard to various embodiments of the invention can be implemented using software, hardware, and combinations of software and hardware. As such, the illustrated and/or described modules and circuitry are intended to encompass software implementations, hardware implementations, and software and hardware implementations.

All publications and patent applications in this specification are indicative of the level of ordinary skill in the art to which this invention pertains. All publications and patent applications are herein incorporated by reference to the same extent as if each individual publication or patent application was specifically and individually indicated by reference.

This application is intended to cover adaptations or variations of the present subject matter. It is to be understood that the above description is intended to be illustrative, and not restrictive. The scope of the present subject matter should be determined with reference to the appended claims, along with the full scope of equivalents to which such claims are entitled.

We claim:

1. A method comprising:
displaying parameters associated with a medical device on a user interface;
receiving data comprising: medical device data from the medical device and an override parameter;
displaying a notification that the override parameter is inconsistent with patient indications; and
prompting user for an override rationale associated with the override parameter on the user interface.

2. The method of claim 1, wherein receiving data further comprises receiving patient symptom data.

3. The method of claim 1, wherein receiving data further comprises receiving electronic medical record data.

4. The method of claim 1, further comprising providing an indication of system confidence level.

5. The method of claim 1, further comprising identifying conflicting data based on at least one of the override rationale and the override parameter.

6. The method of claim 5, further comprising excluding conflicting data from system processes.

7. The method of claim 5, further comprising displaying data accuracy guidance.

8. The method of claim 5, further comprising suppressing reporting an indication based on conflicting data for a period of time.

9. The method of claim 1, wherein options for the override rationale are presented to a user, the options reflecting: physician preference; accuracy of sensor data; accuracy of electronic medical record data; and patient symptom data.

10. The method of claim 1, further comprising receiving the override rationale and incorporating the override rationale in system operations.

11. The method of claim 10, further comprising storing the override rationale and the override parameter and providing a reminder of the override rationale and the override parameter upon future system operations.

12. The method of claim 10, wherein the override rationale is a free text entry by a system user.

13. The method of claim 1, wherein displaying parameters associated with a medical device further comprises displaying system-recommended parameters associated with the medical device.

14. A system comprising:
a programming device in communication with an implantable medical device, an implantable sensor, and electronic medical records, wherein the programming device is configured to program the implantable medical device; and
a user interface in communication with the programming device, wherein the user interface is configured to receive an override parameter and override rationale.

15. The system of claim 14, wherein the programming device is configured to incorporate the override parameter and override rationale in system operations.

16. The system of claim 14 further comprising a display in communication with the programmer configured to display a notice of patient indications inconsistent with the override parameter.

17. The system of claim 15, wherein system operations comprise at least one of:
displaying system-recommended parameters associated with the medical device, excluding conflicting data based on the override rationale, and providing new system-recommended parameters.

18. The system of claim 14, wherein the user interface is configured to receive override rationale that is a free text entry by a system user.

19. The system of claim 14, wherein the programming device is configured to provide a reminder of the override parameter and the override rationale upon future system operations.
